SEQUENCE LISTING (5 sequences)

SEQ ID NO: 1 - DNA, 2349 bp, synthetic construct
  misc_feature 1..2349: note = Synthetic: hVAC14opt
  source 1..2349: mol_type = other DNA
atgaaccctgagaaggactttgcccccctgacccctaacatagtcagagccttgaatgacaagctctatgaaaagaggaaggtggctgctctggagattgaaaaacttgtgagagaatttgtggcccaaaacaacacagtgcagatcaagcatgtcatccagactctgagccaggagtttgccctgagccagcaccctcacagcagaaagggtggactgattggcctggctgcctgtagcattgcccttggaaaggattcaggattgtacctcaaagaactgattgagcctgtcctgacttgctttaatgatgcagactccagactgagatactatgcctgtgaagccctctacaacattgtcaaggtggccagaggtgctgtgttgccccatttcaatgtgttgtttgatggactgtccaagctggctgctgaccctgaccctaatgtgaagtcaggctcagaactgctggacaggctgttgaaggacattgtgactgaatccaataagtttgatttggtgtcattcatccccttgcttagggaaaggatttactccaacaaccagtatgccagacagttcatcattagctggattctggtgctggagtcagtgcctgatattaaccttctggattatctgccagaaatccttgatggcctgttccagattcttggagataatggaaaggagattagaaagatgtgtgaagtggtactgggggaattcctgaaggaaatcaagaagaacccctcctcagtcaagtttgctgagatggccaacatccttgtgatccactgtcagaccactgatgacctgatccaactgactgccatgtgctggatgagggaattcatccagcttgcaggaagagtcatgctgccttactcatcaggtattctcactgctgtgcttccctgtttggcctatgatgatagaaagaagtccattaaggaagtggccaatgtgtgtaaccagtcccttatgaagctggtgacccctgaagatgatgagttagatgaacttaggcctggacagaggcaggctgagcccactccagatgatgccctgcccaagcaagaggggacagccagtggaggacctgatggaagctgtgactcctccttctcctctggcatttcagtgtttactgcagcctccactgaaagggctcctgtcaccctccacctggatggtattgtccaggtgctgaactgtcacctgtctgatacagccattggcatgatgactagaattgcagtcctgaagtggctgtaccacctctacatcaaaactcccaggaagatgttcaggcatactgatagcctcttcccaatcttgctgcagaccctctcagatgaatcagatgaagtgatcctcaaggatctggaggtgctggctgaaattgcctcaagccctgcaggccagacagatgaccctggccccctggatggacctgatctgcaagccagccactcagaacttcaggtgccaacccctggaagggctggactcctgaacacttcaggaaccaaggggctggagtgctcccctagcacacctaccatgaacagctacttctacaagttcatgatcaaccttctcaagagattctcctcagaaagaaagctgttggaggttagaggccctttcatcatcaggcagctctgcctgctcctgaatgctgaaaacattttccactccatggctgatattttgctgagagaagaggacctgaagtttgcctccaccatggtgcatgccctgaacaccatcctcctgacctccactgagctttttcaattgagaaatcagcttaaggaccttaagactctggaatcccagaacctcttttgctgtctgtacagatcctggtgtcacaacccagtcaccactgtgtccctgtgttttctcacccagaattacaggcatgcatatgacctcatccagaagtttggagacttggaggtcactgtggacttcctggctgaggtggacaaactggtgcaactgatagagtgccctatcttcacttacctgaggttgcagctcttggatgtgaaaaacaacccctacctgatcaaggccctgtatggactgttgatgctgctccctcaaagctcagccttccaactcctcagccataggctccagtgtgtgcccaacccagagcttctgcaaactgaggacagccttaaggcagcccccaagtcccaaaaggctgactccccctcaattgactatgctgagctcttacaacactttgaaaaggtgcagaacaagcatctggaagtcagacaccagagaagtggcaggggagatcatctggacagaagggtggtgctctga

SEQ ID NO: 2 - DNA, 168 bp, synthetic construct
  misc_feature 1..168: note = Synthetic: 5' ITR
  source 1..168: mol_type = other DNA
tagctgcgcgctcgctcgctcactgaggccgcccgggcaaagcccgggcgtcgggcgacctttggtcgcccggcctcagtgagcgagcgagcgcgcagagagggagtggccaactccatcactaggggttccttgtagttaatgattaacccgccatgctacttatct

SEQ ID NO: 3 - DNA, 326 bp, synthetic construct
  misc_feature 1..326: note = Synthetic: Promoter
  source 1..326: mol_type = other DNA
gggcggagttagggcggagccaatcagcgtgcgccgttccgaaagttgccttttatggctgggcggagaatgggcggtgaacgccgatgattatataaggacgcgccgggtgtggcacagctagttccgtcgcagccgggatttgggtcgcggttcttgtttgtggatccctgtgatcgtcacttggtaagtcactgactgtctatgcctgggaaagggtgggcaggagatggggcagtgcaggaaaagtggcactatgaaccctgcagccctaggaatgcatctagacaattgtactaaccttcttctctttcctctcctgacag

SEQ ID NO: 4 - DNA, 225 bp, synthetic construct
  misc_feature 1..225: note = Synthetic: Poly-A
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaacagcaggcatgctggggatgcggtgggctctatgg

SEQ ID NO: 5 - DNA, 153 bp, synthetic construct
  misc_feature 1..153: note = Synthetic: 3' ITR
  source 1..153: mol_type = other DNA
aggaacccctagtgatggagttggccactccctctctgcgcgctcgctcgctcactgaggccgggcgaccaaaggtcgcccgacgccggccctttgggccggcggcctcagtgagcgagcgagcgcgcagagagggagtggccaagatggagt

(19) United States
(12) Patent Application Publication
(10) Pub. No.: US 2025/0228977 A1
(43) Pub. Date: Jul. 17, 2025

(54) Compositions and Methods for Treating Striatonigral Degeneration

(71) Applicants: Texas Scottish Rite Hospital for Children, Dallas, TX (US); Board of Regents, The University of Texas System, Austin, TX (US)

(72) Inventors: Jonathan J. Rios, Dallas, TX (US); Steven James Gray, Southlake, TX (US); HyeRi Kang, Prosper, TX (US); Bruce Beutler, Irving, TX (US)

(21) Appl. No.: 19/023,736

(22) Filed: Jan. 16, 2025

Related U.S. Application Data
(60) Provisional application No. 63/621,293, filed on Jan. 16, 2024.

(51) Int. Cl. A61K 48/00; A61K 9/00; A61K 38/17; A61P 25/14; C12N 15/86

(52) U.S. Cl. CPC A61K 48/0058; A61K 9/0085; A61K 38/1709; A61K 48/0075; A61K 48/0083; A61P 25/14; C12N 15/86; C12N 2750/14143

(57) ABSTRACT

Provided herein are compositions and methods for treating striatonigral degeneration, and more particularly, to a recombinant adenovirus associated (AAV) vector comprising a nucleic acid comprising in a 5′ to 3′ direction: a 5′ AAV inverted terminal repeat (ITR) sequence, a promoter sequence, a gene encoding a full-length human VAC14 gene, a polyadenylation sequence, and a 3′ ITR sequence.

### CROSS-REFERENCE TO RELATED APPLICATIONS

This application claims priority to U.S. Provisional Application Ser. No. 63/621,293, filed Jan. 16, 2024, the entire contents of which are incorporated herein by reference.

### STATEMENT OF FEDERALLY FUNDED RESEARCH

This invention was made with government support under 1R01AI125581-01 awarded by the National Institutes of Health/NSF/DARPA. The government has certain rights in the invention.

### TECHNICAL FIELD

The present invention relates in general to the field of compositions and methods for treating striatonigral degeneration, and more particularly, to novel adenoviral constructs and methods for treating striatonigral degeneration.

### INCORPORATION-BY-REFERENCE OF MATERIALS FILED ON COMPACT DISC

The present application includes a sequence listing which has been submitted electronically in .XML format via Patent Center and is hereby incorporated by reference in its entirety. Said .XML copy, created on Jan. 15, 2025, is named “TSRH-1046.xml” and is 10,295 bytes in size. The sequence listing contained in this .XML file is part of the specification and is hereby incorporated by reference herein in its entirety.

### BACKGROUND

Without limiting the scope of the disclosure, its background is described in connection with striatonigral degeneration.

The VAC14 protein is part of a multi-protein complex that regulates intracellular concentrations of a rare signaling lipid. Disruption of this complex leads to dysregulation of the lipid and subsequent formation of vacuoles (holes) in cells, leading to cell death. Cells of the central nervous system (brain, nerves, spinal cord) are particularly sensitive to disruption of the cellular concentrations of this signaling lipid. In mice, loss of the Vac14 gene leads to formation of holes in nervous system tissues, significant neurodegeneration, and early death.

Thus, what is needed are novel compositions and methods that overcome mutations in the VAC14 gene to correctly form the multi-protein complex in central nervous system tissues in patients with diseases caused by recessive mutations in the VAC14 gene, such as, striatonigral degeneration.

### SUMMARY OF THE INVENTION

As embodied and broadly described herein, an aspect of the present disclosure relates to a recombinant adenovirus associated (AAV) vector comprising a nucleic acid comprising in a 5′ to 3′ direction: a 5′ AAV inverted terminal repeat (ITR) sequence, a promoter sequence, a gene encoding a full-length human VAC14 gene, a polyadenylation sequence, and a 3′ ITR sequence. In one aspect, the AAV vector is AAV1, AAV2, AAV3, AAV4, AAV5, AAV6, AAV7, AAV8, AAV9, or a variant of any of the foregoing. In another aspect, the 5′ ITR is ITR2m and the 3′ITR is ITR2. In another aspect, the AAV ITRs are AAV2 ITRs, AAV3 ITRs, AAV4 ITRs, AAV5 ITRs, AAV6 ITRs, AAV7 ITRs, AAV8 ITRs, or AAV9 ITRs. In another aspect, the promoter is a constitutive promoter. In another aspect, the promoter is a central nervous system promoter. In another aspect, the promoter is a JeT or a UsP promoter. In another aspect, the full-length human VAC14 gene is codon optimized or a derivative thereof. In another aspect, the 5′ ITR sequence is SEQ ID NO:2, the promoter is SEQ ID NO: 3, the nucleic acid encoding the full-length human VAC14 gene is SEQ ID NO:1, and polyA signal sequence is SEQ ID NO: 4, and the 3′ ITR sequence is SEQ ID NO:5. In another aspect, the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1.

As embodied and broadly described herein, an aspect of the present disclosure relates to a pharmaceutical composition comprising a recombinant adenovirus associated (AAV) vector comprising a nucleic acid comprising in a 5′ to 3′ direction: a 5′ AAV inverted terminal repeat (ITR) sequence, a promoter sequence, a gene encoding a full-length human VAC14 gene, a polyadenylation sequence, and a 3′ ITR sequence. In one aspect, the AAV vector is AAV1, AAV2, AAV3, AAV4, AAV5, AAV6, AAV7, AAV8, AAV9, or a variant of any of the foregoing. In another aspect, the 5′ ITR is ITR2m and the 3′ITR is ITR2. In another aspect, the AAV ITRs are AAV2 ITRs, AAV3 ITRs, AAV4 ITRs, AAV5 ITRs, AAV6 ITRs, AAV7 ITRs, AAV8 ITRs, or AAV9 ITRs. In another aspect, the promoter is a constitutive promoter. In another aspect, the promoter is a central nervous system promoter. In another aspect, the promoter is a JeT or a UsP promoter. In another aspect, the full-length human VAC14 gene is codon optimized or a derivative thereof. In another aspect, the 5′ ITR sequence is SEQ ID NO:2, the promoter is SEQ ID NO: 3, the nucleic acid encoding the full-length human VAC14 gene is SEQ ID NO:1, and polyA signal sequence is SEQ ID NO: 4, and the 3′ ITR sequence is SEQ ID NO:5. In another aspect, the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1.

As embodied and broadly described herein, an aspect of the present disclosure relates to a method to treat a subject with a diseases associated with a VAC14 gene mutation comprising administering a therapeutically effective amount of a recombinant adenovirus associated (AAV) vector comprising a nucleic acid comprising in a 5′ to 3′ direction: a 5′ AAV inverted terminal repeat (ITR) sequence, a promoter sequence, a gene encoding a full-length human VAC14 gene, a polyadenylation sequence, and a 3′ ITR sequence, and/or the pharmaceutical composition of the same, to the subject, to thereby increase expression of full-length human VAC14 gene in a central nervous system tissue of the subject. In one aspect, the e administration is systemic or intrathecal. In another aspect, a single dose is administered to the subject. In another aspect, the administration is by intravenous infusion. In another aspect, the dose administered is from about in an amount of from about 1×10⁸ to 1×10¹⁵ vector genomes (vg) per kg of body weight of the subject (vg/kg), about 1×10¹⁰ to 1×10¹² vg/kg, 1×10¹³ vg/kg to about 1×10¹⁴ vg/kg, about 1×10¹⁴ vg/kg to 1×10¹⁵ vg/kg, about 2×10¹⁴ vg/kg, 3×10¹³ vg/kg, 4×10¹³ vg/kg, 5×10¹³ vg/kg, 6×10¹³ vg/kg, 7×10¹³ vg/kg, 8×10¹³ vg/kg, 9×10¹⁴, or 1×10¹⁵ vg/kg. In another aspect, the total dose administered is from about 1×10¹² to 1×10¹⁸ total vector genomes (vg) dosed to the subject, about 1×10¹² to 1×10¹⁷ vg, 1×10¹³ vg to about 1×10¹⁶ vg, about 1×10¹⁴ vg to 1×10¹⁵ vg, about 2×10¹⁶ vg, 3×10¹⁶ vg, 4×10¹⁶ vg, 5×10¹⁶ vg, 6×10¹⁶ vg, 7×10¹⁶ vg, 8×10¹⁶ vg, 9×10¹⁶, or 1×10¹⁷ total vg. In another aspect, one or more of the following occur in the subject following administration: reduce or eliminate motor function deficits, reduce or eliminate dystonia, or both. In another aspect, the subject is a pediatric subject with Childhood-Onset Striatonigral Degeneration, Yunis-Varon Syndrome, or a sudden onset of a progressive neurological disorder and regression of developmental milestones.

As embodied and broadly described herein, an aspect of the present disclosure relates to a recombinant adenovirus (AAV) vector encoding a promoter and a full-length human VAC14 gene which has a nucleotide sequence shown in SEQ ID NO: 1. In one aspect, the AAV vector is AAV1, AAV2, AAV3, AAV4, AAV5, AAV6, AAV7, AAV8, AAV9, or a variant of any of the foregoing. In another aspect, the promoter is a constitutive promoter. In another aspect, the promoter is a central nervous system promoter. In another aspect, the full-length human VAC14 gene is codon optimized. In another aspect, the 5′ ITR sequence is SEQ ID NO:2, the promoter is SEQ ID NO: 3, the nucleic acid encoding the full-length human VAC14 gene is SEQ ID NO:1, and polyA signal sequence is SEQ ID NO: 4, and the 3′ ITR sequence is SEQ ID NO:5. In another aspect, the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1.

As embodied and broadly described herein, an aspect of the present disclosure relates to a vector comprising a synthetic nucleic acid that encodes a recombinant adenovirus associated (AAV) vector comprising a nucleic acid comprising in a 5′ to 3′ direction: a 5′ AAV inverted terminal repeat (ITR) sequence, a promoter sequence, a gene encoding a full-length human VAC14 gene, a polyadenylation sequence, and a 3′ ITR sequence. In one aspect, the vector is a viral vector.

As embodied and broadly described herein, an aspect of the present disclosure relates to a recombinant adenovirus associated (AAV) vector comprising in its genome: (a) a 5′ AAV inverted terminal repeat (ITR) and a 3′ AAV ITR; (b) located between the 5′ITR and 3′ITR, a nucleic acid encoding at least 80% identity to SEQ ID NO: 1, operatively linked to a promoter that expresses the nucleic acid in the central nerve system. In one aspect, the recombinant AAV vector is a chimeric AAV vector, haploid AAV vector, a hybrid AAV vector, or polyploid AAV vector. In another aspect, the recombinant AAV vector is any AAV serotype. In another aspect, the serotype is AAV9. In another aspect, the nucleic acid has at least 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1.

As embodied and broadly described herein, an aspect of the present disclosure relates to a pharmaceutical composition comprising a recombinant adenovirus associated (AAV) vector comprising in its genome: (a) a 5′ AAV inverted terminal repeat (ITR) and a 3′ AAV ITR; (b) located between the 5′ITR and 3′ITR, a nucleic acid encoding at least 80% identity to SEQ ID NO: 1 in a pharmaceutically acceptable carrier.

As embodied and broadly described herein, an aspect of the present disclosure relates to a method of increasing Vac14 gene expression in a subject in need thereof, comprising: administering to the subject a therapeutically effective amount of the recombinant AAV vector and a pharmaceutical composition, wherein an optimized nucleic acid is expressed in the subject, thereby overcoming loss-of-function or hypomorphic mutations of the Vac14 gene. In another aspect, the subject has or is at risk for developing childhood-onset striatonigral degeneration, Yunis-Varon Syndrome, or a sudden onset of a progressive neurological disorder and regression of developmental milestones.

As embodied and broadly described herein, an aspect of the present disclosure relates to an AAV9 vector comprising a 5′ inverted terminal repeat (ITR) sequence, a UsP promoter, a codon-optimized hVAC14 coding sequence (hVAC14opt), a polyadenylation sequence, and 5′ AAV inverted terminal repeat (ITR) sequence.

### BRIEF DESCRIPTION OF THE DRAWINGS

For a more complete understanding of the features and advantages of the present disclosure, reference is now made to the detailed description of the disclosure along with the accompanying figures and in which:

FIGS. 1A to 1C shows the CNS vacuolization in Vac14^{ducky} mice. (FIG. 1A) Representative images of 4-month old male and female control (Vac14^{+/+}) and Vac 14^{ducky} mice. (FIG. 1B) Schematic of the Vac 14^{ducky} locus and identification of aberrant splice transcripts expressed in brain tissues of Vac14^{ducky} mice. (FIG. 1C) Relative expression of properly-spliced Vac14 transcript in brain tissue from control and Vac14^{ducky} mice. Data represent mean and s.e.m. from n=3 mice per group. Statistically significant differences were measured by 2-sided T-test. Representative H/E staining of the brain (scale bar=3 mm), brain cortex (scale bar=60 μm), and thoracic dorsal root ganglia (DRG; scale bar=100 μm) from 4-month old control (Vac14^{+/+}) and Vac 14^{ducky} mice. Star indicates DRG. SC, spinal cord.

FIGS. 2A to 2K show that gene replacement therapy rescues motor function in Vac 14^{ducky} mice. (FIG. 2A) Representative images of Vac14^{ducky} mouse embryonic fibroblasts (MEFs) expressing mCherry negative control or the full-length human VAC14 protein-coding transcript (hVAC14). (FIG. 2B) Schematic of the AAV9/hVAC14opt vector. (FIG. 2C) Schematic of the pre-clinical study design utilizing Vac14^{ducky} mice. (FIG. 2D) Representative H/E sections of the brain cortex and thoracic DRG from post-natal day 7 (P7) Vac14^{ducky} mice. Star indicates DRG. SC, spinal cord. (FIG. 2E-FIG. 2G) Longitudinal (FIG. 2E) body weight, (FIG. 2F) forelimb grip strength, and (FIG. 2G) rotarod performance of control (Vac14^{+/+}) and Vac 14^{ducky} mice treated with either vehicle-only negative control or AAV9/hVAC14opt. Significant differences were determined by 2-way ANOVA with Tukey multiple test correction. *, p<0.05; **, p<0.01; ***, p<0.001. (FIG. 2H) Images following tail suspension of 4-month old Vac14^{ducky} mice treated with vehicle negative control (top) or AAV9/hVAC14opt therapy (bottom). (FIG. 2I) Representative H/E staining (left) and localization of VAC14 protein (right) in the brain cortex, spinal cord (SC), and thoracic DRG of 4-month old Vac14^{ducky} mice treated with vehicle negative control or AAV9/hVAC14opt. Scale bars represent 1 mm. (cortex) or 500 μm (DRG). Star indicates DRG. (FIG. 2J) Quantification of vacuole size in the brain cortex of 4-month old Vac14^{ducky} mice treated with vehicle negative control or AAV9/hVAC14opt. Data are from 2 mice per group with group means shown by blue bar. Significant differences were determined using the Mann-Whitney test. ****, p<0.0001. In the brain cortex, the mean size of vacuoles in AAV9/hVAC14opt-treated Vac14^{ducky} mice was significantly less than vehicle treated Vac 14^{ducky} mice (FIG. 2K), which correlated with more cells with detectable VAC14 expression in AAV9/hVAC14opt-treated Vac14^{ducky} mice compared to vehicle treated Vac 14^{ducky} mice (see inset of FIG. 2J).

FIGS. 3A to 3D are graphs that show mice that were evaluated up to 1-year of age to test the durability of the AAV9/hVAC14opt therapy in Vac14^{ducky} mice and to test for detectable toxicity in treated control mice. No significant differences were detected between control mice and AAV9/hVAC14opt-treated Vac14^{ducky} mice out to 1-year of age, suggesting the AAV9/hVAC14opt therapy is durable to later adulthood in mice (FIG. 3A-C). Likewise, survival was significantly rescued in AAV9/hVAC14opt-treated Vac 14^{ducky} mice compared to vehicle-treated Vac 14^{ducky} mice (FIG. 3D). As well, no significant differences were observed in control mice treated with vehicle or AAV9/hVAC14opt, suggesting the AAV9/hVAC14opt therapy is tolerated even at high dose (FIG. 3A-D).

FIGS. 4A to 4D are graphs that show a therapeutically relevant dose. Vac 14^{ducky} mice were treated with vehicle or a human-equivalent target dose of AAV9/hVAC14opt (1×; 2.55×10¹¹ vector genomes). Mice were evaluated out to 1-year of age. Similar to results using a high-dose (4×) treatment with AAV9/hVAC14opt, a single P7 administration of a therapeutic dose (1×) of AAV9/hVAC14opt to Vac14^{ducky} mice significantly improved survival, body weight, grip strength, and rotarod performance compared to Vac14^{ducky} mice treated with vehicle (FIG. 4A-D). No significant differences were detected between Vac14^{ducky} mice treated with a therapeutic (1×) dose of AAV9/hVAC14opt and control mice. These results suggest the AAV9/hVAC14opt therapy is effective and with durable therapeutic response when administered at the human-equivalent target dose.

### DETAILED DESCRIPTION OF THE INVENTION

While the making and using of various aspects of the present disclosure are discussed in detail below, it should be appreciated that the present disclosure provides many applicable inventive concepts that can be embodied in a wide variety of specific contexts. The specific aspects discussed herein are merely illustrative of specific ways to make and use the disclosure and do not delimit the scope of the disclosure.

To facilitate the understanding of this disclosure, a number of terms are defined below. Terms defined herein have meanings as commonly understood by a person of ordinary skill in the areas relevant to the present disclosure. Terms such as “a”, “an” and “the” are not intended to refer to only a singular entity, but include the general class of which a specific example may be used for illustration. The terminology herein is used to describe specific aspects of the disclosure, but their usage does not delimit the disclosure, except as outlined in the claims.

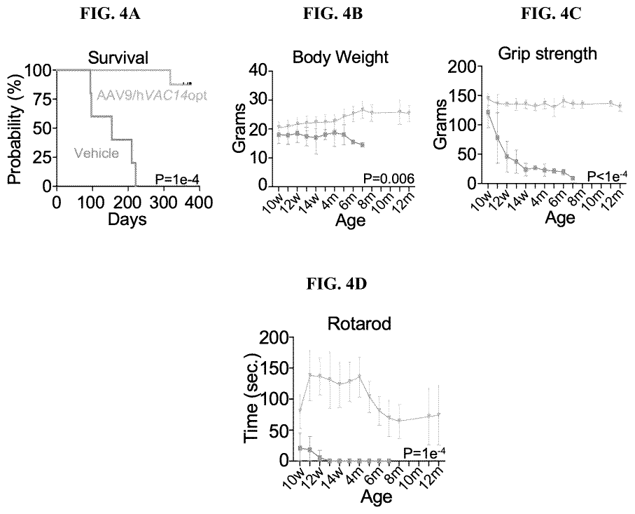

In humans, heterozygous mutations in the PIKFYVE gene cause Mouchetée-type corneal fleck dystrophy, while recessive mutations in VAC14 cause, e.g., SNDC and other conditions, and mutations in FIG. 4 cause Charcot-Marie-Tooth disease type 4J (CMT-4J) (3, 5, 6). SNDC is a severe pediatric neurodegenerative disease that develops in the first half-decade of life, progresses rapidly, and results in spasticity, dystonia, and loss of speech (7). Treatment options for SNDC are limited and generally ineffective in preventing or delaying disease progression. The neurodegenerative clinical manifestations associated with loss of VAC14 or FIG. 4 reinforce the critical role for PI (3,5) P2 homeostasis in CNS development and myelination (8). The present invention can be used to treat any disease in which a mutation of VAC14 is involved.

As used herein, the phrases “a diseases associated with a VAC14 gene mutation” or “diseases associated with VAC14 gene mutations” refer to that/those disease(s) that are caused alone or in part by a mutation of the VAC14 gene. Non-limiting examples of diseases associated with VAC14 gene mutations include Childhood-Onset Striatonigral Degeneration, Yunis-Varon Syndrome, or a sudden onset of a progressive neurological disorder and regression of developmental milestones (See e.g., Lenk, et. al, “Biallelic Mutations of VAC14 in Pediatric-Onset Neurological Disease”, Am J Hum Genet,. 2016 Jul. 7; 99 (1): 188-94. doi: 10.1016/j.ajhg.2016.05.008. Epub 2016 Jun. 9), each of which can be treated at least partially with the present invention.

The present inventors developed and tested a novel gene therapy for the treatment of SNDC in pediatric patients. Gene therapy utilizes a safe virus to infect cells of the nervous system with the goal of delivering a normal copy of the gene to all the infected cells. The inventors show herein the delivery of a normal copy of the human codon-optimized VAC14 gene into the central nervous system of diseased mice (those with mutations in Vac14).

As used herein, the term “codon-optimized” refers to a coding sequence that is optimized relative to a wild type coding sequence (e.g., a coding sequence for VAC14) to reduce CpG content versus the normal human gene. CpG reduction was used to provide a safety advantage because it reduces Tol9 Receptor stimulation during vector trafficking and thus reduces or eliminated any immune response against the vector DNA. Making the VAC14 sequence different from the normal human gene sequence also aids in tracking the vector DNA (distinct from host human DNA) in manufacturing, preclinical studies, and clinical studies by various molecular methods. In additional examples, the codon-optimization substitutions can minimize rare codons (e.g., human codons), decrease total GC content, remove cryptic splice donor or acceptor sites, and/or add or remove ribosomal entry sites, such as Kozak sequences. International PCT Publication No. WO 2017/218450 (relevant portions incorporated herein by reference) discloses codon-optimized of gene sequences, methods for producing, and general methods for delivery of a monotherapy using the codon-optimized genes.

The diseased mice treated with the gene therapy of the present invention do not develop any significant neurodegenerative traits compared to untreated diseased mice, tested using a variety of techniques. It was found that treatment of normal mice with the gene therapy caused no detectable toxicities associated with the gene therapy treatment. As such, the present inventors have developed a gene therapy product that provides a one-time treatment cure for SNDC.

As used herein, the term “nucleic acid” refers to an oligomer or polymer (a linear polymer) of any length composed essentially of nucleotides. A nucleotide unit commonly includes a heterocyclic base, a sugar group, and at least one, e.g. one, two, or three, phosphate groups, including modified or substituted phosphate groups. Heterocyclic bases may include inter alia purine and pyrimidine bases such as adenine (A), guanine (G), cytosine (C), thymine (T) and uracil (U) which are widespread in naturally occurring nucleic acids, other naturally-occurring bases (e.g., xanthine, inosine, hypoxanthine) as well as chemically or biochemically modified (e.g., methylated), non-natural or derivatized bases. Sugar groups may include inter alia pentose (pentofuranose) groups such as preferably ribose and/or 2-deoxyribose common in naturally occurring nucleic acids, or arabinose, 2-deoxyarabinose, threose or hexose sugar groups, as well as modified or substituted sugar groups. Nucleic acids as intended herein may include naturally occurring nucleotides, modified nucleotides, or mixtures thereof. A modified nucleotide may include a modified heterocyclic base, a modified sugar moiety, a modified phosphate group or a combination thereof. Modifications of phosphate groups or sugars may be introduced to improve stability, resistance to enzymatic degradation, or some other useful property. The term “nucleic acid” further preferably encompasses DNA, RNA and DNA RNA hybrid molecules, specifically including hnRNA, pre-mRNA, mRNA, cDNA, genomic DNA, amplification products, oligonucleotides, and synthetic (e.g., chemically synthesized) DNA, RNA or DNA RNA hybrids. A nucleic acid can be naturally occurring, e.g., present in or isolated from nature; or can be non-naturally occurring, e.g., recombinant, i.e., produced by recombinant DNA technology, and/or partly or entirely, chemically or biochemically synthesized. A “nucleic acid” can be double-stranded, partly double stranded, or single-stranded. If single-stranded, the nucleic acid can be the sense strand or the antisense strand. In addition, nucleic acid can be circular or linear.

As used herein, the terms “identity” and “identical” refer to the sequence similarity between two polymeric molecules, e.g., between two nucleic acid molecules, such as between two DNA molecules. Sequence alignments and determination of sequence identity can be done, e.g., using the Basic Local Alignment Search Tool (BLAST) originally described by, e.g., Altschul et al. 1990 (J Mol Biol 215:403-10), such as the “Blast 2 sequences” algorithm described by Tatusova and Madden 1999 (FEMS Microbiol Lett 174:247-250). Methods for aligning sequences for comparison are well-known in the art. Various programs and alignment algorithms are described in, for example: Smith and Waterman (1981) Adv. Appl. Math. 2:482; Needleman and Wunsch (1970) J. Mol. Biol. 48:443; Pearson and Lipman (1988) Proc. Natl. Acad. Sci. U.S.A. 85:2444; Higgins and Sharp (1988) Gene 73:237-44; Higgins and Sharp (1989) CABIOS 5:151-3; Corpet et al. (1988) Nucleic Acids Res. 16:10881-90; Huang et al. (1992) Comp. Appl. Biosci. 8:155-65; Pearson et al. (1994) Methods Mol. Biol. 24:307-31; Tatiana et al. (1999) FEMS Microbiol. Lett. 174:247-50. A detailed consideration of sequence alignment methods and homology calculations can be found in, e.g., Altschul et al. (1990) J. Mol. Biol. 215:403-10, relevant portions incorporated herein by reference.

A National Center for Biotechnology Information (NCBI) Basic Local Alignment Search Tool (BLAST™; Altschul et al. (1990)) is available from several sources, including the National Center for Biotechnology Information (Bethesda, MD), and on the internet, for use in connection with several sequence analysis programs. A description of how to determine sequence identity using this program is available on the internet under the “help” section for BLAST™. For comparisons of nucleic acid sequences, the “Blast 2 sequences” function of the BLAST™ (Blastn) program may be employed using the default parameters. Nucleic acid sequences with even greater similarity to the reference sequences will show increasing percentage identity when assessed by this method. Typically, the percentage sequence identity is calculated over the entire length of the sequence. For example, a global optimal alignment is suitably found by the Needleman-Wunsch algorithm with the following scoring parameters: Match score: +2, Mismatch score: −3; Gap penalties: gap open 5, gap extension 2. The percentage identity of the resulting optimal global alignment is suitably calculated by the ratio of the number of aligned bases to the total length of the alignment, where the alignment length includes both matches and mismatches, multiplied by 100.

As used herein, the term “hybridizing” refers to the annealing to two at least partially complementary nucleotide sequences in a hybridization process. In order to allow hybridization to occur complementary nucleic acid molecules are generally thermally or chemically denatured to melt a double strand into two single strands and/or to remove hairpins or other secondary structures from single-stranded nucleic acids. The stringency of hybridization is influenced by conditions such as temperature, salt concentration and hybridization buffer composition. Conventional hybridization conditions are described in, for example, Sambrook (2001) MOLECULAR CLONING: A LABORATORY MANUAL, 3rd Edition Cold Spring Harbor Laboratory Press, CSH, New York, but the skilled craftsman will appreciate that numerous different hybridization conditions can be designed in function of the known or the expected homology and/or length of the nucleic acid sequence. High stringency conditions for hybridization include high temperature and/or low sodium/salt concentration (salts include sodium as for example in NaCl and Na-citrate) and/or the inclusion of formamide in the hybridization buffer and/or lowering the concentration of compounds such as SDS (sodium dodecyl sulphate detergent) in the hybridization buffer and/or exclusion of compounds such as dextran sulphate or polyethylene glycol (promoting molecular crowding) from the hybridization buffer. By way of non-limiting example, representative salt and temperature conditions for stringent hybridization are: 1×SSC, 0.5% SDS at 65° C. The abbreviation SSC refers to a buffer used in nucleic acid hybridization solutions. One liter of a 20× (twenty times concentrate) stock SSC buffer solution (pH 7.0) contains 175.3 g sodium chloride and 88.2 g sodium citrate. A representative time period for achieving hybridization is 1 to 12 hours.

As used herein, the term “synthetic” refers to a nucleic acid molecule that does not occur in nature. Synthetic nucleic acid expression constructs of the present invention are produced artificially, typically by recombinant technologies. Such synthetic nucleic acids may contain naturally occurring sequences (e.g., promoter(s), enhancer(s), intron(s), and other such regulatory sequence(s)), but these are present in a non-naturally occurring context. For example, a synthetic gene (or portion of a gene) typically contains one or more nucleic acid sequences that are not contiguous in nature (chimeric sequences), and/or may encompass substitutions, insertions, and deletions and combinations thereof. As used herein, the term “synthetic promoter” relates to a promoter that does not occur in nature.

As used herein, the terms “complementary” or “complementarity” refers to the Watson-Crick base-pairing of two nucleic acid sequences. For example, for the sequence 5′-AGT-3′ binds to the complementary sequence 3′-TCA-5′. Complementarity between two nucleic acid sequences may be “partial”, in which only some of the bases bind to their complement, or it may be complete as when every base in the sequence binds to its complementary base. The degree of complementarity between nucleic acid strands has significant effects on the efficiency and strength of hybridization between nucleic acid strands.

As used herein, the terms “spacer sequence” or “spacer” refer to a nucleic acid sequence that separates two functional nucleic acid sequences. It can have essentially any sequence, provided it does not prevent the functional nucleic acid sequence (e.g., cis-regulatory element) from functioning as desired (e.g., if it includes a silencer sequence, prevents binding of the desired transcription factor, or suchlike). Typically, it is non-functional, as in it is present only to space adjacent functional nucleic acid sequences from one another.

As used herein, the term “amino acid” refers to any naturally occurring amino acid, modified forms thereof, and synthetic amino acids.

As used herein, the term “vector” refers to a compound used as a vehicle to carry foreign genetic material into another cell, where the genetic material can be replicated and/or expressed. A cloning vector containing foreign nucleic acid is termed a recombinant vector. Examples of nucleic acid vectors are plasmids, viral vectors, cosmids, and artificial chromosomes. Recombinant vectors typically contain an origin of replication, a multicloning site, and a selectable marker. The nucleic acid sequence typically consists of an insert (recombinant nucleic acid or transgene) and a larger sequence that serves as the “backbone” of the vector. The purpose of a vector which transfers genetic information to another cell is typically to isolate, multiply, or express the insert in the target cell. Expression vectors (expression constructs) are for the expression of the exogenous gene in the target cell, and generally have a promoter sequence that drives expression of the exogenous gene or other nucleic acid. Insertion of a vector into the target cell is referred to transformation or transfection for bacterial and eukaryotic cells, although insertion of a viral vector is often called transduction. As used herein, the term “vector” may also be used in general to describe items that serve to carry foreign genetic material into another cell, such as, but not limited to, a transformed cell or a nanoparticle.

As used herein, the term “delivery vectors” refers to constructs that deliver their nucleic acid cargo into a cell, typically to express the nucleic acid in the cell. In one embodiment, delivery vectors of the present invention include, without limitation viral vectors. A variety of viral vectors are known in the art (e.g., those derived from adenoviruses, herpesvirus, Epstein-Barr virus, retrovirus, baculovirus, adenovirus, or parvovirus such as adeno-associated virus). Non-viral delivery vectors are also known in the art and their use is also encompassed by the instant invention. In one embodiment, the viral vector is a recombinant adeno-associated virus (AAV). Such viral vectors comprise an AAV capsid and can package an AAV or rAAV genome or any other nucleic acid including viral nucleic acids. Alternatively, in some contexts, the term “vector,” “virus vector,” “delivery vector” (and similar terms) may be used to refer to the vector genome (e.g., vDNA) in the absence of the virion and/or to a viral capsid that acts as a transporter to deliver molecules tethered to the capsid or packaged within the capsid.

As used herein, the terms “virus vector,” “AAV vector,” or “viral delivery vector” generally refer to a virus particle that functions as a nucleic acid delivery vehicle, and which comprises the viral nucleic acid (i.e., the vector genome) packaged within the virion. Examples of viral vectors of the invention include “recombinant AAV vector genome” or “rAAV genome”, such as, any AAV genome (i.e., vDNA) that comprises at least one inverted terminal repeat (e.g., one, two or three inverted terminal repeats) and one or more heterologous nucleotide sequences . . . rAAV vectors generally retain the 145 base inverted terminal repeat(s) (ITR(s)) in cis to generate virus; however, modified AAV TRs and non-AAV TRs including partially, or completely, synthetic sequences can also serve this purpose. All other viral sequences are dispensable and may be supplied in trans (Muzyczka, (1992) Curr. Topics Microbiol. Immunol. 158:97). The rAAV vector optionally comprises two ITRs (e.g., AAV ITRs), which generally will be at the 5′ and 3′ ends of the heterologous nucleotide sequence(s), but need not be contiguous thereto. The ITRs can be the same or different from each other. The vector genome can also contain a single ITR at its 3′ or 5′ end.

As used herein, the terms “virus vector,” “viral vector”, “vector” or “gene delivery vector” refer to a virus (e.g., AAV) particle that functions as a nucleic acid delivery vehicle, and which comprises the vector genome (e.g., viral DNA [vDNA]) packaged within a virion.

As used herein, the term “viral vector” may refer to a nucleic acid vector construct that includes at least one element of viral origin and has the capacity to be packaged into a viral vector particle. The viral vector can contain a nucleic acid encoding a polypeptide as described herein in place of non-essential viral genes. The vector and/or particle may be utilized for the purpose of transferring synthetic nucleic acids described herein into cells either in vitro or in vivo. Numerous forms of viral vectors are known in the art and provided herein.

As used herein, an “rAAV vector genome”, “AAV vector”, or “rAAV genome” is an AAV genome (i.e., viral DNA (vDNA)) that comprises one or more heterologous nucleic acid sequences . . . rAAV vectors generally require only the inverted terminal repeat(s) (TR(s)) in cis to generate virus. All other viral sequences are dispensable and may be supplied in trans (see, e.g., Muzyczka, (1992) Curr. Topics Microbial. Immunol. 158:97). Generally, the rAAV vector genome will only retain the one or more TR sequences that maximize the size of the transgene that can be efficiently packaged by the vector. The structural and non-structural protein coding sequences may be provided in trans (e.g., from a vector, such as a plasmid, or by stably integrating the sequences into a packaging cell). In embodiments of the invention the rAAV vector genome comprises at least one ITR sequence (e.g., AAV TR sequence), optionally two ITRs (e.g., two AAV TRs), which typically will be at the 5′ and 3′ ends of the vector genome and flank the heterologous nucleic acid, but need not be contiguous thereto. The TRs can be the same or different from each other.

As used herein, the terms “viral genome” or “vg” refer to viral units provided for gene therapy and are typically noted as vg/kg of the subject or total vg dose per patient. The experiments described herein are amounts that were used to treat mice, and it is inferred that when the subject being treated is not a mouse, the amount is converted to an amount that is appropriate for the subject being treated, e.g., a human, an infant or newborn. In one aspect, the amount administered is in vg/kg and therefore accounts for difference in size and body weight of the subject being treated. In another aspect, the dose can be a total vg dose per subject, which can be scaled across species and/or subjects by other means such as cerebrospinal fluid volume or brain size.

The AAV vector may be administered to the patient in a therapeutically effective amount, such as in an amount of from about 1×10¹² to 1×10¹⁵ vector genomes (vg) per kg of body weight of the subject (vg/kg) to about 1×10¹⁴ vg/kg (e.g., in an amount of from about 1×10¹³ vg/kg to about 1×10¹⁵ vg/kg, such as in an amount of from about 1×10¹³ vg/kg to about 2×10¹⁴ vg/kg, 3×10¹³ vg/kg, 4×10¹³ vg/kg, 5×10¹³ vg/kg, 6×10¹³ vg/kg, 7×10¹³ vg/kg, 8×10¹³ vg/kg, 9×10¹⁴ 1×10¹⁵ vg/kg. Likewise, the total vg dose administered to the subject in a therapeutically effective amount, such as in an amount of from about 1×10¹² to 1×10¹⁸ total vector genomes (vg) dosed in one or more doses, about 1×10¹² to 1×10¹⁷ vg, 1×10¹³ vg to about 1×10¹⁶ vg, about 1×10¹⁴ vg to 1×10¹⁵ vg, about 2×10¹⁶ vg, 3×10¹⁶ vg, 4×10¹⁶ vg, 5×10¹⁶ vg, 6×10¹⁶ vg, 7×10¹⁶ vg, 8×10¹⁶ vg, 9×10¹⁶, or 1×10¹⁷ vg.

In some embodiments, the AAV vector is administered to the patient in a single dose, which in other embodiments, the AAV vector is administered in one or more doses that each, individually, contain the specified amount. For example, the AAV vector may be administered to the patient in from one to ten doses that each, individually, contain the specified amount (e.g., in two, three, four, five, six, seven, eight, nine, or ten doses that each, individually, contain the specified amount). In some embodiments, the AAV vector is administered to the patient in two, three, or four doses that each, individually, contain the specified amount. In some embodiments, the AAV vector is administered to the patient in two doses that each, individually, contain the specified amount.

In some embodiments, the AAV vector is administered to the patient by way of intravenous, intrathecal, intracisternal, intracerebroventricular, intramuscular, intradermal, transdermal, parenteral, intranasal, subretinal, intravitreal, intranerve, subcutaneous, percutaneous, intratracheal, intraperitoneal, intraarterial, intravascular, inhalation, perfusion, lavage, and/or oral administration. For example, the AAV vector may be administered to the patient by way of intravenous, intrathecal, intracisternal, intracerebroventricular, and/or intramuscular administration. In some embodiments, the AAV vector is administered to the patient by way of intravenous and/or intrathecal administration . . . some embodiments, the AAV vector is administered to the patient by way of intravenous administration. The AAV vector may also be administered by any combination of these routes of administration, simultaneously or sequentially.

As used herein, the term “recombinant expression system” or “recombinant vector” refers to a genetic construct or constructs for the expression of certain genetic material formed by recombination.

As used herein, the term “terminal repeat” or “TR” refers to any viral terminal repeat or synthetic sequence that forms a hairpin structure and functions as an inverted terminal repeat (i.e., an ITR that mediates the desired functions such as replication, virus packaging, integration and/or provirus rescue, and the like).

As used herein, an “AAV terminal repeat” or “AAV TR,” including an “AAV inverted terminal repeat” or “AAV ITR” may be from any AAV, including but not limited to serotypes 1, 2, 3, 4, 5, 6, 7, 8, 9, 10, 11 or 12 or any other AAV now known or later discovered. An AAV terminal repeat need not have the native terminal repeat sequence (e.g., a native AAV TR or AAV ITR sequence may be altered by insertion, deletion, truncation and/or missense mutations), if the terminal repeat mediates the desired functions, e.g., replication, virus packaging, integration, and/or provirus rescue, and the like. AAV proteins VP1, VP2 and VP3 are capsid proteins that interact together to form an AAV capsid of an icosahedral symmetry. VP1.5 is an AAV capsid protein described in US Publication No. 2014/0037585. The capsid proteins can be naturally occurring or modified, as is well known in the art. Further, the viral capsid or genomic elements can contain other modifications, including insertions, deletions and/or substitutions.

As used herein, a “chimeric’ capsid protein refers to an AAV capsid protein that has been modified by substitutions in one or more (e.g., 2, 3, 4, 5, 6, 7, 8, 9, 10, etc.) amino acid residues in the amino acid sequence of the capsid protein relative to wild type, as well as insertions and/or deletions of one or more (e.g., 2, 3, 4, 5, 6, 7, 8, 9, 10, etc.) amino acid residues in the amino acid sequence relative to wild type. In some embodiments, complete or partial domains, functional regions, epitopes, etc., from one AAV serotype can replace the corresponding wild type domain, functional region, epitope, etc. of a different AAV serotype, in any combination, to produce a chimeric capsid protein of this invention. Production of a chimeric capsid protein can be carried out according to protocols well known in the art and a significant number of chimeric capsid proteins are described in the literature as well as herein that can be included in the capsid of this invention.

As used herein, the term “cis-regulatory element” or “CRE”, is a term well-known to the skilled person, and refers to a nucleic acid sequence such as an enhancer, promoter, insulator, or silencer, that can regulate or modulate the transcription of an adjacent gene (i.e. in cis). CREs are found in the vicinity of the genes that they regulate. CREs typically regulate gene transcription by binding to TFs, i.e., they include TFBS. A single TF may bind to many CREs, and hence control the expression of many genes (pleiotropy). CREs are usually, but not always, located upstream of the transcription start site (TSS) of the gene that they regulate. “Enhancers” are CREs that enhance (i.e., upregulate) the transcription of genes that they are operably associated with, and can be found upstream, downstream, and even within the introns of the gene that they regulate. Multiple enhancers can act in a coordinated fashion to regulate transcription of one gene. “Silencers” in this context relates to CREs that bind TFs called repressors, which act to prevent or downregulate transcription of a gene.

As used herein, the term “silencer” refers to a region in the 3′ untranslated region of messenger RNA, that bind proteins which suppress translation of that mRNA molecule, but this usage is distinct from its use in describing a CRE. Generally, the CREs of the present invention are central nervous system-specific enhancers (often referred to as central nervous system-specific CREs, or central nervous system-specific CRE enhancers, or suchlike). In the present context, the CRE can be located 1500 nucleotides or less from the transcription start site (TSS), 1000 nucleotides or less from the TSS, 500 nucleotides or less from the TSS, and generally 250, 200, 150, or 100 nucleotides or less from the TSS. CREs are preferably comparatively short in length, preferably 100 nucleotides or less in length, for example they may be 90, 80, 70, 60 nucleotides or less in length.

As used herein, the term “cis-regulatory module” or “CRM” refers to a functional module made up of two or more CREs; in the present invention the CREs are typically liver-specific enhancers. Thus, in the present application a CRM typically comprises one or more central nervous system-specific enhancer CREs. Generally, the multiple CREs within the CRM act together (e.g., additively or synergistically) to enhance the transcription of a gene that the CRM is operably associated with. There is conservable scope to shuffle (i.e., reorder), invert (i.e., reverse orientation), and alter spacing in CREs within a CRM. Accordingly, functional variants of CRMs of the present invention include variants of the referenced CRMs wherein CREs within them have been shuffled and/or inverted, and/or the spacing between CREs has been altered.

As used herein, the phrase “promoter” refers to a region of DNA that generally is located upstream of a nucleic acid sequence to be transcribed that is needed for transcription to occur, i.e., which initiates transcription. Promoters permit the proper activation or repression of transcription of a coding sequence under their control. A promoter typically contains specific sequences that are recognized and bound by plurality of TFs. TFs bind to the promoter sequences and result in the recruitment of RNA polymerase, an enzyme that synthesizes RNA from the coding region of the gene. A great many promoters are known in the art.

As used herein, the term “synthetic promoter” refers to a promoter that does not occur in nature. In the present context it typically comprises a synthetic CRE and/or CRM of the present invention operably linked to a minimal (or core) promoter or proximal promoter, e.g., a central nervous system-specific. The CREs and/or CRMs of the present invention serve to enhance central nervous system-specific transcription of a gene operably linked to the promoter. Parts of the synthetic promoter may be naturally occurring (e.g., the minimal promoter or one or more CREs in the promoter), but the synthetic promoter as a complete entity is not naturally occurring. As used herein, a ubiquitously expressed UsP promoter is a short synthetic and ubiquitous promoter derived from the JeT promoter and a synthetic intron (UsP, also referred to as the JeTI promoter.

As used herein, the terms “minimal promoter” or “core promoter” refer to a short DNA segment that is inactive or largely inactive by itself but can mediate transcription when combined with other transcription regulatory elements. Minimum promoter sequence can be derived from various sources, including prokaryotic and eukaryotic genes. Examples of minimal promoters are discussed above and include the dopamine beta-hydroxylase gene minimum promoter, cytomegalovirus (CMV) immediate early gene minimum promoter (CMV-MP), and the herpes thymidine kinase minimal promoter (MinTK). A minimal promoter typically comprises the transcription start site (TSS) and elements directly upstream, a binding site for RNA polymerase II, and general transcription factor binding sites (often a TATA box).

As used herein, the term “central nervous system specific promoter” refers to those sequences that promote substantially higher expression in central nervous system tissue than other tissues. Examples of central nervous system specific promoters include, without limitation, synapsin I promoter (SYN1 or hSYN), glial fibrillary acidic protein promoter (GFAP), internexin neuronal intermediate filament protein, alpha promoter or α-internexin promoter (INA), nestin promoter (NES), myelin-associated oligodendrocyte basic protein promoter (MOBP), myelin basic protein promoter (MBP), myelin protein zero (MPZ) promoter, tyrosine hydroxylase promoter (TH) and forkhead box A2 promoter (FOXA₂).

As used herein, the terms “central nervous system-specific” or “central nervous system-specific expression” refer to the ability of a cis-regulatory element, cis-regulatory module or promoter to enhance or drive expression of a gene in central nervous system tissue (or in central nervous system-derived cells) in a preferential or predominant manner as compared to other tissues (e.g., spleen, liver, lung, blood, and muscle). Expression of the gene can be in the form of mRNA or protein. In some embodiments, a central nervous system specific promoter promotes expression in tissues of the central nervous system, including those that express VAC14. In some embodiments, the central nervous system specific promoter promotes 30% or more, 40% or more, 50% or more, 60% or more, 70% or more, 80% or more, 90% or more, 95% or more, 96% or more, 97% or more, 98% or more, 99% or more expression in central nervous system tissue than one or more other tissues. In some embodiments, the central nervous system specific promoter results in no significant or detectable expression in one or more non-central nervous system tissues.

As used herein, the term “pharmaceutically acceptable” refers to those ingredients compatible with the other ingredients of the pharmaceutical composition and not deleterious to the recipient thereof.

As used herein, the terms “administer” or “administration” refers to the delivery of a substance to a subject such as an animal or human. Administration can be in a single dose. However, if two or more doses are used, the additional doses may be administered continuously or intermittently throughout the course of treatment. Methods of determining the most effective means and dosage of administration are known to those of skill in the art and may vary with the composition used for therapy, the purpose of the therapy, as well as the age, health or gender of the subject being treated. Single or multiple administrations can be carried out with the dose level and pattern being selected by the treating physician. In the case of pets and animals by the treating veterinarian. Suitable dosage formulations and methods of administering the agents are known in the art, with certain examples described hereinabove. The route and method of determining the most effective route of administration are known to those of skill in the art and will vary with the composition used for treatment, the purpose of the treatment, the health condition or disease stage of the subject being treated and the target cell or tissue. Non-limiting examples of route of administration of the present invention include intravenous, intrathecal, intra-arterial, intramuscular, intracardiac, subventricular, epidural, intracerebral, intracerebroventricular, sub-retinal, intravitreal, intranerve, intraarticular, intraocular, intraperitoneal, intrauterine, intradermal, subcutaneous, transdermal, transmucosal, and inhalation.

As used herein, the term “effective amount” is synonymous with “therapeutically effective amount”, “effective dose”, or “therapeutically effective dose.” Thus, a “therapeutically effective” amount refers to an amount that is sufficient to provide some improvement or benefit to the subject, or is an amount that will provide some alleviation, mitigation, decrease or stabilization in at least one clinical symptom in the subject. Those skilled in the art will appreciate that the therapeutic effects need not be complete or curative, if some benefit is provided to the subject. In an embodiment, the effectiveness of a therapeutic compound disclosed herein to treat VAC14-associated named conditions, such as, SNDC, can be determined, without limitation, by observing an improvement in an individual based upon one or more clinical symptoms, and/or physiological indicators associated with the disorder. In an embodiment, an improvement in the symptoms associated with the disorder can be indicated by a reduced need for a concurrent therapy.

As used herein, the term “prevention effective” amount as used herein is an amount that is sufficient to prevent and/or delay the onset of SNDC and/or clinical symptoms in a subject and/or to reduce and/or delay the severity of the onset of a disease, disorder and/or clinical symptoms in a subject relative to what would occur in the absence of the methods of the invention. Those skilled in the art will appreciate that the level of prevention need not be complete, as long as some benefit is provided to the subject.

### Model and Gene Therapy for VAC14-Associated SNDC.

Phosphatidylinositol 3,5-bisphosphate [PI (3,5) P2] is a low abundance signaling lipid with diverse cellular functions (1). Intracellular PI (3,5) P2 concentrations are regulated by a multi-protein complex including the scaffold protein VAC14, the FIG. 4 phosphoinositide 5-phosphatase, and the FYVE-type zinc finger containing phosphoinositide kinase (PIKFYVE, also known as FAB1). The importance of maintaining PI (3,5) P2 homeostasis, particularly in neuronal tissues, is evidenced by the neurodegeneration and lethality associated with PI (3,5) P2 deficiency. For example, mice homozygous for loss-of-function or hypomorphic mutations in Vac14, or the pale tremor (plt) mouse line harboring a loss-of-function mutation in FIG. 4, develop extensive vacuolization of neuronal tissues and exhibit neurodegenerative sequelae and pre-weaning lethality, while mice lacking Pikfyve are embryonic lethal (2-4). To date, no mouse line harboring disease-causing mutations in this protein complex survive to adulthood.

The gene therapy vector of the present invention targets a genetic deficiency that causes childhood-onset striatonigral degeneration (www.omim.org/entry/617054), which is caused by mutations in the VAC14 gene. Patients inherit two mutations in the VAC14 gene (NCBI Gene: 55697 (transcript NM_018052.5), OMIM®: 604632, UniProtKB/Swiss-Prot: Q08AM6) that results in a complete loss or insufficient amount of normal protein.

hVAC14opt-
SEQ ID NO: 1
ATGAACCCTGAGAAGGACTTTGCCCCCCTGACCCCTAACATAGTC
AGAGCCTTGAATGACAAGCTCTATGAAAAGAGGAAGGTGGCTGCT
CTGGAGATTGAAAAACTTGTGAGAGAATTTGTGGCCCAAAACAAC
ACAGTGCAGATCAAGCATGTCATCCAGACTCTGAGCCAGGAGTTT
GCCCTGAGCCAGCACCCTCACAGCAGAAAGGGTGGACTGATTGGC
CTGGCTGCCTGTAGCATTGCCCTTGGAAAGGATTCAGGATTGTAC
CTCAAAGAACTGATTGAGCCTGTCCTGACTTGCTTTAATGATGCA
GACTCCAGACTGAGATACTATGCCTGTGAAGCCCTCTACAACATT
GTCAAGGTGGCCAGAGGTGCTGTGTTGCCCCATTTCAATGTGTTG
TTTGATGGACTGTCCAAGCTGGCTGCTGACCCTGACCCTAATGTG
AAGTCAGGCTCAGAACTGCTGGACAGGCTGTTGAAGGACATTGTG
ACTGAATCCAATAAGTTTGATTTGGTGTCATTCATCCCCTTGCTT
AGGGAAAGGATTTACTCCAACAACCAGTATGCCAGACAGTTCATC
ATTAGCTGGATTCTGGTGCTGGAGTCAGTGCCTGATATTAACCTT
CTGGATTATCTGCCAGAAATCCTTGATGGCCTGTTCCAGATTCTT
GGAGATAATGGAAAGGAGATTAGAAAGATGTGTGAAGTGGTACTG
GGGGAATTCCTGAAGGAAATCAAGAAGAACCCCTCCTCAGTCAAG
TTTGCTGAGATGGCCAACATCCTTGTGATCCACTGTCAGACCACT
GATGACCTGATCCAACTGACTGCCATGTGCTGGATGAGGGAATTC
ATCCAGCTTGCAGGAAGAGTCATGCTGCCTTACTCATCAGGTATT
CTCACTGCTGTGCTTCCCTGTTTGGCCTATGATGATAGAAAGAAG
TCCATTAAGGAAGTGGCCAATGTGTGTAACCAGTCCCTTATGAAG
CTGGTGACCCCTGAAGATGATGAGTTAGATGAACTTAGGCCTGGA
CAGAGGCAGGCTGAGCCCACTCCAGATGATGCCCTGCCCAAGCAA
GAGGGGACAGCCAGTGGAGGACCTGATGGAAGCTGTGACTCCTCC
TTCTCCTCTGGCATTTCAGTGTTTACTGCAGCCTCCACTGAAAGG
GCTCCTGTCACCCTCCACCTGGATGGTATTGTCCAGGTGCTGAAC
TGTCACCTGTCTGATACAGCCATTGGCATGATGACTAGAATTGCA
GTCCTGAAGTGGCTGTACCACCTCTACATCAAAACTCCCAGGAAG
ATGTTCAGGCATACTGATAGCCTCTTCCCAATCTTGCTGCAGACC
CTCTCAGATGAATCAGATGAAGTGATCCTCAAGGATCTGGAGGTG
CTGGCTGAAATTGCCTCAAGCCCTGCAGGCCAGACAGATGACCCT
GGCCCCCTGGATGGACCTGATCTGCAAGCCAGCCACTCAGAACTT
CAGGTGCCAACCCCTGGAAGGGCTGGACTCCTGAACACTTCAGGA
ACCAAGGGGCTGGAGTGCTCCCCTAGCACACCTACCATGAACAGC
TACTTCTACAAGTTCATGATCAACCTTCTCAAGAGATTCTCCTCA
GAAAGAAAGCTGTTGGAGGTTAGAGGCCCTTTCATCATCAGGCAG
CTCTGCCTGCTCCTGAATGCTGAAAACATTTTCCACTCCATGGCT
GATATTTTGCTGAGAGAAGAGGACCTGAAGTTTGCCTCCACCATG
GTGCATGCCCTGAACACCATCCTCCTGACCTCCACTGAGCTTTTT
CAATTGAGAAATCAGCTTAAGGACCTTAAGACTCTGGAATCCCAG
AACCTCTTTTGCTGTCTGTACAGATCCTGGTGTCACAACCCAGTC
ACCACTGTGTCCCTGTGTTTTCTCACCCAGAATTACAGGCATGCA
TATGACCTCATCCAGAAGTTTGGAGACTTGGAGGTCACTGTGGAC
TTCCTGGCTGAGGTGGACAAACTGGTGCAACTGATAGAGTGCCCT
ATCTTCACTTACCTGAGGTTGCAGCTCTTGGATGTGAAAAACAAC
CCCTACCTGATCAAGGCCCTGTATGGACTGTTGATGCTGCTCCCT
CAAAGCTCAGCCTTCCAACTCCTCAGCCATAGGCTCCAGTGTGTG
CCCAACCCAGAGCTTCTGCAAACTGAGGACAGCCTTAAGGCAGCC
CCCAAGTCCCAAAAGGCTGACTCCCCCTCAATTGACTATGCTGAG
CTCTTACAACACTTTGAAAAGGTGCAGAACAAGCATCTGGAAGTC
AGACACCAGAGAAGTGGCAGGGGAGATCATCTGGACAGAAGGGTG
GTGCTCTGA
5' ITR-
SEQ ID NO: 2
TAGCTGCGCGCTCGCTCGCTCACTGAGGCCGCCCGGGCAAAGCCC
GGGCGTCGGGCGACCTTTGGTCGCCCGGCCTCAGTGAGCGAGCGA
GCGCGCAGAGAGGGAGTGGCCAACTCCATCACTAGGGGTTCCTTG
TAGTTAATGATTAACCCGCCATGCTACTTATCT
Promoter-
SEQ ID NO: 3
GGGCGGAGTTAGGGCGGAGCCAATCAGCGTGCGCCGTTCCGAAAG
TTGCCTTTTATGGCTGGGCGGAGAATGGGCGGTGAACGCCGATGA
TTATATAAGGACGCGCCGGGTGTGGCACAGCTAGTTCCGTCGCAG
CCGGGATTTGGGTCGCGGTTCTTGTTTGTGGATCCCTGTGATCGT
CACTTGGTAAGTCACTGACTGTCTATGCCTGGGAAAGGGTGGGCA
GGAGATGGGGCAGTGCAGGAAAAGTGGCACTATGAACCCTGCAGC
CCTAGGAATGCATCTAGACAATTGTACTAACCTTCTTCTCTTTCC
TCTCCTGACAG
Poly-A-
SEQ ID NO: 4
CTGTGCCTTCTAGTTGCCAGCCATCTGTTGTTTGCCCCTCCCCCG
TGCCTTCCTTGACCCTGGAAGGTGCCACTCCCACTGTCCTTTCCT
AATAAAATGAGGAAATTGCATCGCATTGTCTGAGTAGGTGTCATT
CTATTCTGGGGGGTGGGGTGGGGCAGGACAGCAAGGGGGAGGATT
GGGAAGACAACAGCAGGCATGCTGGGGATGCGGTGGGCTCTATGG
3' ITR-
SEQ ID NO: 5
AGGAACCCCTAGTGATGGAGTTGGCCACTCCCTCTCTGCGCGCTC
GCTCGCTCACTGAGGCCGGGCGACCAAAGGTCGCCCGACGCCGGC
CCTTTGGGCCGGCGGCCTCAGTGAGCGAGCGAGCGCGCAGAGAGG
GAGTGGCCAAGATGGAGT

Human skin cells cultured from a patient with SNDC showed significant vacuolization that was reversed by transient re-expression of the normal human gene copy (6). The inventors cultured mouse embryonic fibroblast cells harvested from a pre-clinical disease model developed and characterized by the inventors (Vac14^{ducky}). Cultured cells showed significant vacuolization, and when the present invention (hVAC14opt) was expressed in mouse cells, vacuolization was reversed. Thus, expression of the human gene copy via the present invention is sufficient to treat the disease pathology in primary mouse cells.

Mouse model system. Through the course of an N-ethyl-N-nitrosourea (ENU) saturation mutagenesis screen in mice (9-11), several phenovariant mice were detected with diluted coat color, dystonic movements, and reduced lean mass. The phenotype in these mice, termed ducky, showed a recessive inheritance pattern, and pedigree mapping identified a donor splice-site allele within intron 8 of the Vac14 gene (Vac14^{ex8+3A>T}) as the likely culprit mutation. To validate the causality of the ducky allele, CRISPR/Cas9 was used to engineer knock-in mice harboring the ducky splice allele. Again, mice homozogyous for the allele (herein called Vac 14^{ducky}) developed a diluted coat color and dystonia. Phenotypic similarities between the Vac14^{ducky} mice, the previously published ingls mouse line harboring a hypomorphic missense mutation in Vac14 (12), and the previously published plt mouse line (3) are consistent with the ducky allele being a loss-of-function mutation. However, unlike ingls and plt mice, Vac 14^{ducky} mice survive to adulthood despite significant neurodegeneration.

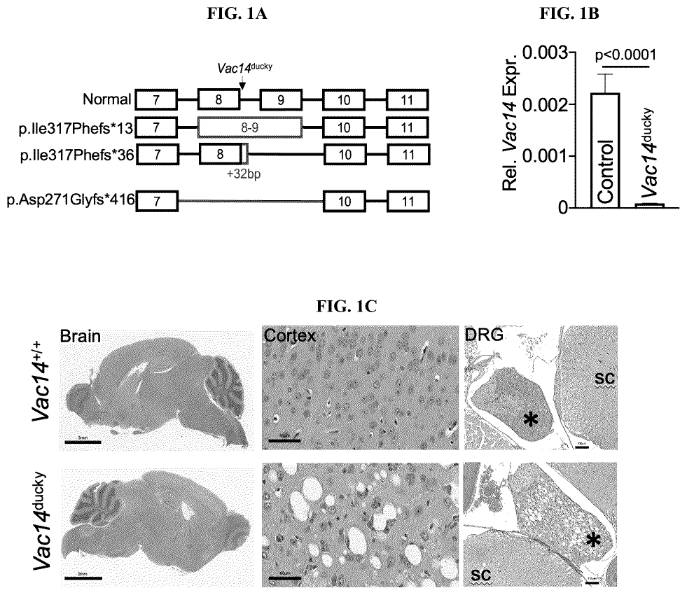

The ducky allele is predicted to alter Vac14 splicing and result in reduced expression of full-length transcript. Three alternatively spliced transcripts expressed in brain tissue were detected from Vac14^{ducky} mice that were not evident in controls, and each was predicted to result in premature truncation (FIG. 1A). Expression of properly spliced Vac14 transcript was significantly reduced, though still detectable, in Vac14^{ducky} brain tissue compared to control (FIG. 1B). In vivo, extensive vacuolization was evident within the brain cortex and thoracic dorsal root ganglia (DRG) of 4 month-old Vac 14^{ducky} mice (FIG. 1C). These results suggest the Vac 14^{ducky} allele is hypomorphic, although with sufficient residual Vac14 expression for survival.

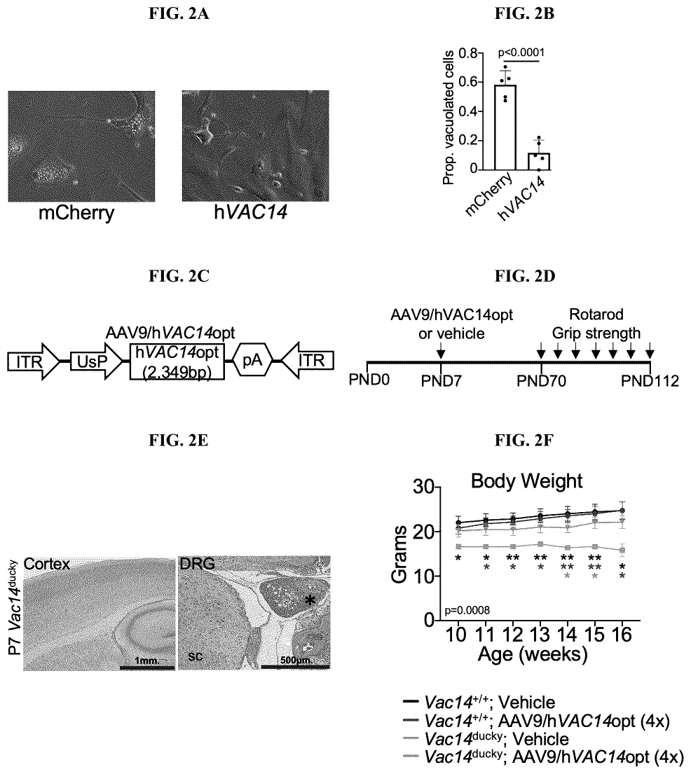

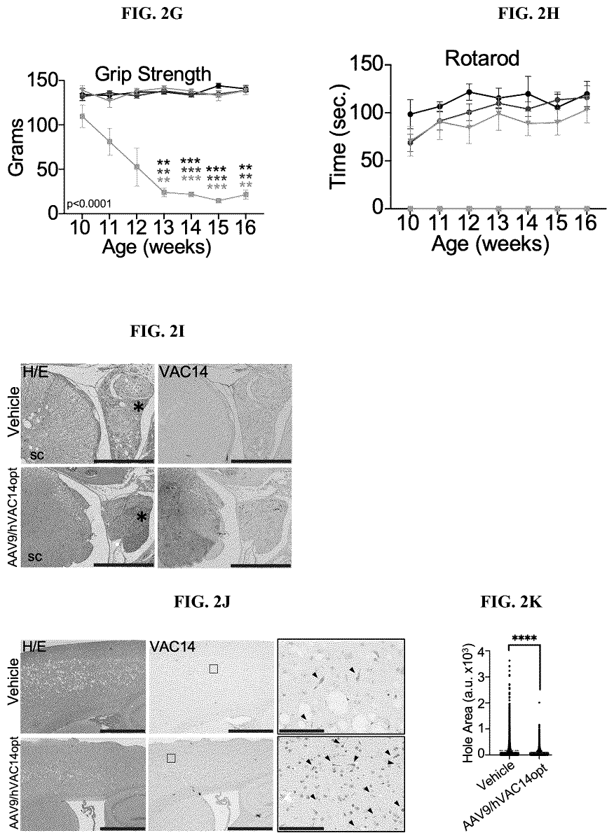

Cultured mouse embryonic fibroblasts (MEFs) from Vac 14^{ducky} mice demonstrated extensive vacuolization compared to control. To test whether vacuolization was reversible, the full-length human VAC14 coding sequence (hVAC14) was expressed in Vac14^{ducky} MEFs, and vacuolization was compared to cells expressing the fluorescent mCherry construct (negative control). Vacuolization of Vac14^{ducky} MEFs was significantly rescued in cells expressing hVAC14 compared to mCherry (FIG. 2A, 2B), similarly to previous experiments using patient fibroblasts (6). These results suggest gene-replacement therapy may rescue vacuolization, and possibly neurodegeneration, in Vac 14^{ducky} mice. A single-stranded AAV9 vector was engineered carrying a codon-optimized hVAC14 coding sequence (hVAC14opt) driven by a ubiquitously expressed UsP promoter (herein called AAV9/hVAC14opt) (FIG. 2C). The UsP promoter is a short synthetic and ubiquitous promoter derived from the JeT promoter and a synthetic intron (UsP, also referred to as the JeTI promoter.

To test the efficacy of hVAC14 gene-replacement therapy, Vac 14^{ducky} mice were treated at post-natal day 7 (P7) with a single intrathecal dose of AAV9/hVAC14opt (4× dose; 1.02×10¹² vector genomes) or vehicle and evaluated weekly from 10-weeks to 4-months of age (FIG. 2D). At P7, vacuolization was present in the DRG but not the brain cortex of Vac14^{ducky} mice (FIG. 2E). As expected, vehicle-treated Vac14^{ducky} mice showed reduced body weight and progressive loss of forelimb grip strength (FIG. 2F,G). Due to their significant dystonia, no vehicle-treated Vac 14^{ducky} mice were able to complete rotarod testing (FIG. 2H). In contrast, body weight, grip strength, and rotarod performance of AAV9/hVAC14opt-treated Vac 14^{ducky} mice were similar to control mice at all timepoints measured (FIG. 2F-H). At 4 months of age, vehicle-treated Vac 14^{ducky} mice demonstrated dystonia-like responses to tail suspension, which were rescued in AAV9/hVAC14opt-treated Vac14^{ducky} mice. These results demonstrate the in vivo efficacy of AAV9/hVAC14opt gene-replacement therapy to rescue motor function deficits and dystonia in Vac 14^{ducky} mice.

Previous studies, including in plt mice, demonstrated efficient targeting of the thoracic DRG via intrathecal AAV9 injection, with significantly less targeting to the brain cortex (13, 14). Therefore, vacuolization in 4-month old vehicle- and AAV9/hVAC14opt-treated Vac 14^{ducky} mice was evaluated. Vacuole formation within the thoracic DRG tissues was dramatically rescued in AAV9/hVAC14opt-treated Vac 14^{ducky} mice compared to vehicle-treated mice, which correlated with the robust production of VAC14 in these mice (FIG. 2I). Similarly, vacuolization was significantly, though not completely, reduced in the brain cortex of Vac 14^{ducky} mice treated with the AAV9/hVAC14opt gene replacement therapy (FIG. 2J). In the brain cortex, the mean size of vacuoles in AAV9/hVAC14opt-treated Vac 14^{ducky} mice was significantly less than vehicle treated Vac 14^{ducky} mice (FIG. 2K), which correlated with more cells with detectable VAC14 expression in AAV9/hVAC14opt-treated Vac14^{ducky} mice compared to vehicle treated Vac 14^{ducky} mice (see inset of FIG. 2J).

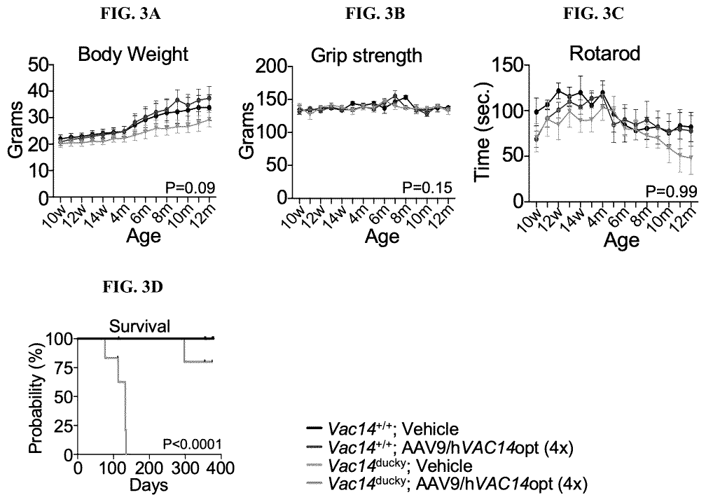

Mice were subsequently evaluated up to 1-year of age to test the durability of the AAV9/hVAC14opt therapy in Vac 14^{ducky} mice and to test for detectable toxicity in treated control mice. No significant differences were detected between control mice and AAV9/hVAC14opt-treated Vac14^{ducky} mice out to 1-year of age, suggesting the AAV9/hVAC14opt therapy is durable to later adulthood in mice (FIG. 3A-C). Likewise, survival was significantly rescued in AAV9/hVAC14opt-treated Vac 14^{ducky} mice compared to vehicle-treated Vac14^{ducky} mice (FIG. 3D). As well, no significant differences were observed in control mice treated with vehicle or AAV9/hVAC14opt, suggesting the AAV9/hVAC14opt therapy is tolerated even at high dose (FIG. 3A-D).

To test a therapeutically relevant dose, Vac 14^{ducky} mice were treated with vehicle or a human-equivalent target dose of AAV9/hVAC14opt (1×; 2.55×10¹¹ vector genomes). Mice were evaluated out to 1-year of age. Similar to results using a high-dose (4×) treatment with AAV9/hVAC14opt, a single P7 administration of a therapeutic dose (1×) of AAV9/hVAC14opt to Vac14^{ducky} mice significantly improved survival, body weight, grip strength, and rotarod performance compared to Vac 14^{ducky} mice treated with vehicle (FIG. 4A-D). No significant differences were detected between Vac 14^{ducky} mice treated with a therapeutic (1×) dose of AAV9/hVAC14opt and control mice. These results suggest the AAV9/hVAC14opt therapy is effective and with durable therapeutic response when administered at the human-equivalent target dose.

These results demonstrate the efficacy of a novel gene-replacement therapy for SNDC using a single dose of the present invention. Direct CNS administration of the AAV9/hVAC14opt vector significantly reduced vacuolization in the affected tissues and rescued motor function performance of Vac14^{ducky} mice. The therapeutic efficacy of AAV9/hVAC14opt reported here, together with prior work in plt mice (14), demonstrate the potential for gene-replacement therapy to treat neurodegenerative disease caused by PI (3,5) P2 deficiency.

### NUMBERED EMBODIMENTS OF THE INVENTION

Notwithstanding the appended claims, the disclosure sets forth the following numbered embodiments:
1. A recombinant adenovirus associated (AAV) vector comprising a nucleic acid comprising in a 5′ to 3′ direction: a 5′ AAV inverted terminal repeat (ITR) sequence, a promoter sequence, a gene encoding a full-length human VAC14 gene, a polyadenylation sequence, and a 3′ ITR sequence.
2. The recombinant AAV vector of embodiment 1, wherein the AAV vector is AAV1, AAV2, AAV3, AAV4, AAV5, AAV6, AAV7, AAV8, AAV9, or a variant of any of the foregoing.
3. The recombinant AAV vector of embodiments 1 or 2, wherein the 5′ ITR is ITR2m and the 3′ ITR is ITR2.
4. The recombinant AAV vector of any one of embodiments 1-3, wherein the AAV ITRs are AAV2 ITRs, AAV3 ITRs, AAV4 ITRs, AAV5 ITRs, AAV6 ITRs, AAV7 ITRs, AAV8 ITRs, or AAV9 ITRs.
5. The recombinant AAV vector of any one of embodiments 1-4, wherein the promoter is a constitutive promoter.
6. The recombinant AAV vector of any one of embodiments 1-5, wherein the promoter is a central nervous system promoter.
7. The recombinant AAV vector of any one of embodiments 1-6, wherein the promoter is a JeT or a UsP promoter.
8. The recombinant AAV vector of any one of embodiments 1-7, wherein the full-length human VAC14 gene is codon optimized or a derivative thereof.
9. The recombinant AAV vector of any one of embodiments 1-9, wherein the 5′ ITR sequence is SEQ ID NO:2, the promoter is SEQ ID NO: 3, the nucleic acid encoding the full-length human VAC14 gene is SEQ ID NO:1, and polyA signal sequence is SEQ ID NO: 4, and the 3′ ITR sequence is SEQ ID NO:5.
10. The recombinant AAV vector of any one of embodiments 1-10, wherein the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1.
11. A pharmaceutical composition comprising the recombinant AAV vector of any one of claims 1-10.
12. A method to treat a subject with a diseases associated with a VAC14 gene mutation comprising administering a therapeutically effective amount of the recombinant AAV vector of any one of claims 1-10, and/or the pharmaceutical composition of claim 11, to the subject, to thereby increase expression of full-length human VAC14 gene in a central nervous system tissue of the subject.
13. The method of embodiment 12, wherein the administration is systemic or intrathecal.
14. The method of embodiments 12 or 13, wherein a single dose is administered to the subject.
15. The method of any one of embodiments 12-14, wherein administration is by intravenous infusion.
16. The method of any one of embodiments 12-15, wherein the dose administered is from about in an amount of from about 1×10⁸ to 1×10¹⁵ vector genomes (vg) per kg of body weight of the subject (vg/kg), about 1×10¹⁰ to 1×10¹² vg/kg, 1×10¹³ vg/kg to about 1×10¹⁴ vg/kg, about 1×10¹⁴ vg/kg to 1×10¹⁵ vg/kg, about 2×10¹⁴ vg/kg, 3×10¹³ vg/kg, 4×10¹³ vg/kg, 5×10¹³ vg/kg, 6×10¹³ vg/kg, 7×10¹³ vg/kg, 8×10¹³ vg/kg, 9×10¹⁴, or 1×10¹⁵ vg/kg.
17. The method of any one of embodiments 12-16, wherein the total dose administered is from about 1×10¹² to 1×10¹⁸ total vector genomes (vg) dosed to the subject, about 1×10¹² to 1×10¹⁷ vg, 1×10¹³ vg to about 1×10¹⁶ vg, about 1×10¹⁴ vg to 1×10¹⁵ vg, about 2×10¹⁶ vg, 3×10¹⁶ vg, 4×10¹⁶ vg, 5×10¹⁶ vg, 6×10¹⁶ vg, 7×10¹⁶ vg, 8×10¹⁶ vg, 9×10¹⁶, or 1×10¹⁷ total vg
18. The method of any one of embodiments 12-17, wherein one or more of the following occur in the subject following administration: reduce or eliminate motor function deficits, reduce or eliminate dystonia, or both.
19. The method of any one of embodiments 12-18, wherein the subject is a pediatric subject with Childhood-Onset Striatonigral Degeneration, Yunis-Varon Syndrome, or a sudden onset of a progressive neurological disorder and regression of developmental milestones.
20. A recombinant adenovirus (AAV) vector encoding a promoter and a full-length human VAC14 gene which has a nucleotide sequence shown in SEQ ID NO: 1.
21. The recombinant AAV vector of embodiment 20, wherein the AAV vector is AAV1, AAV2, AAV3, AAV4, AAV5, AAV6, AAV7, AAV8, AAV9, or a variant of any of the foregoing.
22. The recombinant AAV vector of embodiments 20 or 21, wherein the promoter is a constitutive promoter.
23. The recombinant AAV vector of any one of embodiments 20-22, wherein the promoter is a central nervous system promoter.
24. The recombinant AAV vector of any one of embodiments 20-23, wherein the full-length human VAC14 gene is codon optimized.
25. The recombinant AAV vector of any one of embodiments 19-23, wherein the 5′ ITR sequence is SEQ ID NO:2, the promoter is SEQ ID NO: 3, the nucleic acid encoding the full-length human VAC14 gene is SEQ ID NO:1, and polyA signal sequence is SEQ ID NO: 4, and the 3′ ITR sequence is SEQ ID NO:5.
26. The recombinant AAV vector of any one of embodiments 20-25, wherein the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1.
27. A vector comprising a synthetic nucleic acid of claim 1.
28. The vector of embodiment 27, wherein the vector is a viral vector.
29. A recombinant adenovirus associated (AAV) vector comprising in its genome: (a) a 5′ AAV inverted terminal repeat (ITR) and a 3′ AAV ITR; (b) located between the 5′ITR and 3′TTR, a nucleic acid encoding at least 80% identity to SEQ ID NO: 1, operatively linked to a promoter that expresses the nucleic acid in the central nerve system.
30. The recombinant AAV vector of embodiment 29, wherein the recombinant AAV vector is a chimeric AAV vector, haploid AAV vector, a hybrid AAV vector, or polyploid AAV vector.
31. The recombinant AAV vector of embodiments 29 or 30, wherein the recombinant AAV vector is any AAV serotype.
32. The recombinant AAV vector of any of embodiments 29-31, wherein the serotype is AAV9.
33. The recombinant AAV vector of any of embodiments 29-32, wherein the nucleic acid has at least 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1.
34. A pharmaceutical composition comprising the recombinant AAV vector of any one of claims 29-33 in a pharmaceutically acceptable carrier.
35. A method of increasing Vac14 gene expression in a subject in need thereof, comprising: administering to the subject a therapeutically effective amount of the recombinant AAV vector of any one of claims 1-10, the pharmaceutical composition of claim 11 or 33, wherein an optimized nucleic acid is expressed in the subject, thereby overcoming loss-of-function or hypomorphic mutations of the Vac14 gene.
36. The method of embodiment 35, wherein the subject has or is at risk for developing childhood-onset striatonigral degeneration, Yunis-Varon Syndrome, or a sudden onset of a progressive neurological disorder and regression of developmental milestones.
37. An AAV9 vector comprising a 5′ inverted terminal repeat (ITR) sequence, a UsP promoter, a codon-optimized hVAC14 coding sequence (hVAC14opt), a polyadenylation sequence, and 5′ AAV inverted terminal repeat (ITR) sequence.

It is contemplated that any aspects of the disclosure discussed in this specification can be implemented with respect to any method, kit, reagent, or composition of the disclosure, and vice versa. Furthermore, compositions of the disclosure can be used to achieve methods of the disclosure.

It will be understood that particular aspects described herein are shown by way of illustration and not as limitations of the disclosure. The principal features of this disclosure can be employed in various aspects without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

All publications and patent applications mentioned in the specification are indicative of the level of skill of those skilled in the art to which this disclosure pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In aspects of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the disclosure(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any disclosure(s) in this disclosure. Neither is the “Summary” to be considered a characterization of the disclosure(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure but should not be constrained by the headings set forth herein.

All of the compositions and/or methods disclosed and claimed herein can be made and executed without undue experimentation in light of the present disclosure. While the compositions and methods of this disclosure have been described in terms of preferred aspects, it will be apparent to those of skill in the art that variations may be applied to the compositions and/or methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

For each of the claims, each dependent claim can depend both from the independent claim and from each of the prior dependent claims for each and every claim so long as the prior claim provides a proper antecedent basis for a claim term or element.

### REFERENCES

1. N. Jin, M. J. Lang, L. S. Weisman, Phosphatidylinositol 3,5-bisphosphate: regulation of cellular events in space and time. Biochem Soc Trans 44, 177-184 (2016).
2. O. C. Ikonomov et al., The phosphoinositide kinase PIKfyve is vital in early embryonic development: preimplantation lethality of PIKfyve−/− embryos but normality of PIKfyve+/− mice. J Biol Chem 286, 13404-13413 (2011).
3. C. Y. Chow et al., Mutation of FIG. 4 causes neurodegeneration in the pale tremor mouse and patients with CMT4J. Nature 448, 68-72 (2007).
4. Y. Zhang et al., Loss of Vac14, a regulator of the signaling lipid phosphatidylinositol 3,5-bisphosphate, results in neurodegeneration in mice. Proc Natl Acad Sci USA 104, 17518-17523 (2007).
5. S. Li et al., Mutations in PIP5K3 are associated with Francois-Neetens mouchetee fleck corneal dystrophy. Am J Hum Genet 77, 54-63 (2005).
6. G. M. Lenk et al., Biallelic Mutations of VAC14 in Pediatric-Onset Neurological Disease. Am J Hum Genet 99, 188-194 (2016).
7. P. Kaur, G. S. Bhavani, A. Raj, K. M. Girisha, A. Shukla, Homozygous variant, p. (Arg643Trp) in VAC14 causes striatonigral degeneration: report of a novel variant and review of VAC14-related disorders. J Hum Genet 64, 1237-1242 (2019).
8. Y. A. Mironova et al., PI (3,5) P2 biosynthesis regulates oligodendrocyte differentiation by intrinsic and extrinsic mechanisms. Elife 5 (2016).
9. T. Wang et al., Probability of phenotypically detectable protein damage by ENU-induced mutations in the Mutagenetix database. Nat Commun 9, 441 (2018).
10. T. Wang et al., Real-time resolution of point mutations that cause phenovariance in mice. Proc Natl Acad Sci USA 112, E440-449 (2015).
11. J. J. Rios et al., Germline saturation mutagenesis induces skeletal phenotypes in mice. J Bone Miner Res 10.1002/jbmr.4323 (2021).
12. N. Jin et al., VAC14 nucleates a protein complex essential for the acute interconversion of PI3P and PI (3,5) P (2) in yeast and mouse. Embo J 27, 3221-3234 (2008).
13. R. M. Bailey, A. Rozenberg, S. J. Gray, Comparison of high-dose intracisterna magna and lumbar puncture intrathecal delivery of AAV9 in mice to treat neuropathies. Brain Res 1739, 146832 (2020).
14. M. Presa et al., AAV9-mediated FIG. 4 delivery prolongs life span in Charcot-Marie-Tooth disease type 4J mouse model. J Clin Invest 131 (2021).

What is claimed is:

1. A recombinant adenovirus associated (AAV) vector comprising a nucleic acid comprising in a 5′ to 3′ direction: a 5′ AAV inverted terminal repeat (ITR) sequence, a promoter sequence, a gene encoding a full-length human VAC14 gene, a polyadenylation sequence, and a 3′ ITR sequence.

2. The recombinant AAV vector of claim 1, wherein at least one of:
the AAV vector is AAV1, AAV2, AAV3, AAV4, AAV5, AAV6, AAV7, AAV8, AAV9, or a variant of any of the foregoing;
the 5′ ITR is ITR2m and the 3′ ITR is ITR2; or
the AAV ITRs are AAV2 ITRs, AAV3 ITRs, AAV4 ITRs, AAV5 ITRs, AAV6 ITRs, AAV7 ITRs, AAV8 ITRs, or AAV9 ITRs.

3. The recombinant AAV vector of claim 1, wherein the promoter is a constitutive promoter, a central nervous system promoter, a JeT, or a UsP promoter.

4. The recombinant AAV vector of claim 1, wherein the full-length human VAC14 gene is codon optimized or a derivative thereof.

5. The recombinant AAV vector of claim 1, wherein the 5′ ITR sequence is SEQ ID NO:2, the promoter is SEQ ID NO: 3, the nucleic acid encoding the full-length human VAC14 gene is SEQ ID NO: 1, and polyA signal sequence is SEQ ID NO: 4, and the 3′ ITR sequence is SEQ ID NO:5.

6. The recombinant AAV vector of claim 1, wherein the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1.

7. The recombinant AAV vector of claim 1, further comprising one or more pharmaceutical acceptable excipients.

8. A method to treat a subject with a diseases associated with a VAC14 gene mutation comprising administering a therapeutically effective amount of the recombinant AAV vector of claim 1 to the subject, to thereby increase expression of full-length human VAC14 gene in a central nervous system tissue of the subject.

9. The method of claim 8, wherein the administration is systemic, intrathecal, or intravenous infusion.

10. The method of claim 8, wherein a single dose is administered to the subject.

11. The method of claim 8, wherein the dose administered is from about in an amount of from about 1×10⁸ to 1×10¹⁵ vector genomes (vg) per kg of body weight of the subject (vg/kg), about 1×10¹⁰ to 1×10¹² vg/kg, 1×10¹³ vg/kg to about 1×10¹⁴ vg/kg, about 1×10¹⁴ vg/kg to 1×10¹⁵ vg/kg, about 2×10¹⁴ vg/kg, 3×10¹³ vg/kg, 4×10¹³ vg/kg, 5×10¹³ vg/kg, 6×10¹³ vg/kg, 7×10¹³ vg/kg, 8×10¹³ vg/kg, 9×10¹⁴, or 1×10¹⁵ vg/kg.

12. The method of claim 8, wherein the total dose administered is from about 1×10¹² to 1×10¹⁸ total vector genomes (vg) dosed to the subject, about 1×10¹² to 1×10¹⁷ vg, 1×10¹³ vg to about 1×10¹⁶ vg, about 1×10¹⁴ vg to 1×10¹⁵ vg, about 2×10¹⁶ vg, 3×10¹⁶ vg, 4×10¹⁶ vg, 5×10¹⁶ vg, 6×10¹⁶ vg, 7×10¹⁶ vg, 8×10¹⁶ vg, 9×10¹⁶, or 1×10¹⁷ total vg.

13. The method of claim 8, wherein one or more of the following occur in the subject following administration: reduce or eliminate motor function deficits, reduce or eliminate dystonia, or both.

14. The method of claim 8, wherein the subject is a pediatric subject with Childhood-Onset Striatonigral Degeneration, Yunis-Varon Syndrome, or a sudden onset of a progressive neurological disorder and regression of developmental milestones.

15. A recombinant adenovirus (AAV) vector encoding a promoter and a full-length human VAC14 gene which has a nucleotide sequence shown in SEQ ID NO: 1.

16. The recombinant AAV vector of claim 15, wherein at least one of:
the AAV vector is AAV1, AAV2, AAV3, AAV4, AAV5, AAV6, AAV7, AAV8, AAV9, or a variant of any of the foregoing;
the 5′ ITR is ITR2m and the 3′ ITR is ITR2; or
the AAV ITRs are AAV2 ITRs, AAV3 ITRs, AAV4 ITRs, AAV5 ITRs, AAV6 ITRs, AAV7 ITRs, AAV8 ITRs, or AAV9 ITRs.

17. The recombinant AAV vector of claim 15, wherein the promoter is a constitutive promoter, a central nervous system promoter, a JeT, or a UsP promoter.

18. The recombinant AAV vector of claim 15, wherein the 5′ ITR sequence is SEQ ID NO:2, the promoter is SEQ ID NO: 3, the nucleic acid encoding the full-length human VAC14 gene is SEQ ID NO:1, and polyA signal sequence is SEQ ID NO: 4, and the 3′ ITR sequence is SEQ ID NO: 5.

19. The recombinant AAV vector of claim 15, wherein the coding sequence has at least 81%, 82%, 83%, 84%, 85%, 86%, 87%, 88%, 89%, 90%, 91%, 92%, 93%, 94%, 95%, 96%, 97%, 98%, or 99% identity to SEQ ID NO: 1.

20. A vector comprising a synthetic nucleic acid of claim 1.

21. The vector of claim 20, wherein the vector is a viral vector.

22. A recombinant adenovirus associated (AAV) vector comprising in its genome: (a) a 5′ AAV inverted terminal repeat (ITR) and a 3′ AAV ITR; (b) located between the 5′ITR and 3′ITR, a nucleic acid encoding at least 80% identity to SEQ ID NO: 1, operatively linked to a promoter that expresses the nucleic acid in the central nerve system.

23. The recombinant AAV vector of claim 22, wherein at least one of:
the recombinant AAV vector is a chimeric AAV vector, haploid AAV vector, a hybrid AAV vector, or polyploid AAV vector;
the recombinant AAV vector is any AAV serotype; or
the serotype is AAV9.

24. The recombinant AAV vector of claim 23, wherein the nucleic acid has at least 81%, 82%, 83%, 84%, 85%, 86%, 87%, 88%, 89%, 90%, 91%, 92%, 93%, 94%, 95%, 96%, 97%, 98%, 99%, or 100% identity to SEQ ID NO: 1.

25. A method of increasing Vac14 gene expression in a subject in need thereof, comprising:
administering to the subject a therapeutically effective amount of the recombinant AAV vector of claim 1, wherein an optimized nucleic acid is expressed in the subject, thereby overcoming loss-of-function or hypomorphic mutations of the Vac14 gene.

26. The method of claim 25, wherein the subject has or is at risk for developing childhood-onset striatonigral degeneration, Yunis-Varon Syndrome, or a sudden onset of a progressive neurological disorder and regression of developmental milestones.

27. An AAV9 vector comprising a 5′ inverted terminal repeat (ITR) sequence, a UsP promoter, a codon-optimized hVAC14 coding sequence (hVAC14opt), a polyadenylation sequence, and 5′ AAV inverted terminal repeat (ITR) sequence.
